(19) United States
(12) Patent Application Publication
(10) Pub. No.: US 2023/0381122 A1
(43) Pub. Date: Nov. 30, 2023

(54) APPLICATION OF N1-DIHYDROCAFFEOYL-N10-CAFFEOYLSPERMIDINE IN PREPARATION OF DRUG, HEALTH PRODUCT OR FUNCTIONAL FOOD

(71) Applicant: NINGXIA MEDICAL UNIVERSITY, YINCHUAN (CN)

(72) Inventors: XUEQIN MA, YINCHUAN (CN); YINGLI WANG, YINCHUAN (CN)

(21) Appl. No.: 18/318,192

(22) Filed: May 16, 2023

Related U.S. Application Data
(63) Continuation of application No. PCT/CN2022/135725, filed on Dec. 1, 2022.

(30) Foreign Application Priority Data
May 31, 2022 (CN) 202210610166.X

(51) Int. Cl. A61K 31/165; A61K 9/00; A61P 25/20; A23L 33/105

(52) U.S. Cl. CPC A61K 31/165; A61K 9/0056; A61P 25/20; A23L 33/105

(57) ABSTRACT

An application of N1-dihydrocaffeoyl-N10-caffeoylspermidine in preparation of a drug, health product or functional food is disclosed. It belongs to the technical field of pharmaceutical chemistry. In the dose range of 2-100 mg/kg given to experimental animals, N1-dihydrocaffeoyl-N10-caffeoylspermidine can significantly reduce the number of activities, shorten the sleep latency and prolong the sleep duration of mice with insomnia, and at the same time, it can significantly regulate back the indicator levels of neurotransmitters such as 5-HT, 5-HIAA, GABA and Glu in the brain tissues of mice with insomnia. All of the above confirm that N1-dihydrocaffeoyl-N10-caffeoylspermidine have a sedative hypnotic effect and can be applied for the prevention and treatment of psychiatric disorders such as insomnia, anxiety and depression.

### CROSS REFERENCE TO RELATED APPLICATION

This patent application claims the benefit and priority of Chinese Patent Application No. 202210610166.X filed on May 31, 2022, the disclosure of which is incorporated by reference herein in its entirety as part of the present application.

### TECHNICAL FIELD

The present disclosure relates to the technical field of pharmaceutical chemistry technology, and more specifically, to an application of N1-dihydrocaffeoyl-N10-caffeoylspermidine in preparation of a drug, health product or functional food.

### BACKGROUND ART

Insomnia, as one of the more common illnesses in life, is closely related to people's daily life and work. The occurrence of insomnia is related to various factors such as age, gender and disease, and the incidence rate of insomnia increases with age. In recent years, the social employment pressure is increasing, the economic demand is growing, and people's living habits are also changing. In this social environment, the incidence rate of insomnia is increasing. Insomnia not only leads to restlessness, depression and other emotions in patients, but also leads to the occurrence of various diseases. Therefore, it has become particularly important to develop and find natural drugs to treat insomnia.

Current drugs for clinical treatment of insomnia mainly include barbiturates, benzodiazepines and non-benzodiazepines sedative hypnotics; melatonin drugs; Orexin receptor antagonists; 5-HT2A receptor antagonists; antipsychotics, antidepressants and antiepileptics and the like. All of the above are chemically synthesized drugs, which show relatively many adverse reactions, and may produce resistance and dependency during long-term use. In recent years, some newly discovered active monomers of sedative and hypnotic traditional Chinese medicine have also been used in research to treat and improve sleep disorders related diseases. It has been proved that syringic acid, isoliquiritigenin and the like have a significant improvement effect on sleep disorder. Therefore, it has become a current research hotspot to find active monomer ingredients which have a sedative hypnotic effect and may be used for preventing and treating diseases such as insomnia, anxiety and depression from natural drugs.

N1-dihydrocaffeoyl-N10-caffeoylspermidine is a spermidine compound which is isolated from Chinese wolfberry fruit. At present, fewer studies have been reported on N1-dihydrocaffeoyl-N10-caffeoylspermidine, and none have reported on the biological activity of N1-dihydrocaffeoyl-N10-caffeoylspermidine.

In conclusion, how to provide an application of N1-dihydrocaffeoyl-N10-caffeoylspermidine is a problem that needs to be addressed by those skilled in the art.

### SUMMARY

In view of the above, the present disclosure provides an application of N1-dihydrocaffeoyl-N10-caffeoylspermidine for the preparation of a drug, a health product, or a functional food. In the present disclosure, through a previous animal experiment, N1-dihydrocaffeoyl-N10-caffeoylspermidine is found to have a significant sedative hypnotic effect. Further, it is of great clinical use to develop N1-dihydrocaffeoyl-N10-caffeoylspermidine as drugs or functional foods capable of preventing and treating insomnia, anxiety and depression, or to improve insomnia, anxiety and depression.

N1-dihydrocaffeoyl-N10-caffeoylspermidine, molecular formula C₂₅H₃₃N₃O₆, English full term (E)-3-(3,4-dihydroxyphenyl)-N-(3-((4-(3,4-dihydroxyphenyl) propanamido) butyl) amino) propyl) acrylamide, abbreviated as N1-dihydrocaffeoyl-N10-caffeoylspermidine, with molecular weight 471.24, and its chemical structure formula is shown in formula (1):

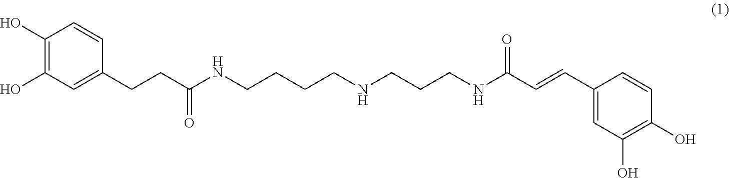 [SMILES: O=C(/C=C/c1ccc(O)c(O)c1)NCCCNCCCCNC(=O)CCc1ccc(O)c(O)c1]

In order to achieve the above purpose, technical solutions of the present disclosure are specifically described as follows:
the application of N1-dihydrocaffeioyl-N10-caffeioylspermidine in a preparation of a drug for preventing and treating insomnia, anxiety and depression, or
the application in a preparation of a health product or a functional food for improving insomnia, anxiety and depression.

Further, the number of autonomous activities of mice with insomnia can be reduced.

Further, the sleep latency can be shortened.

Further, the sleep duration can be prolonged.

Further, the levels of transmitters 5-HT, 5-HIAA, GABA and Glu in brain tissues can be regulated back.

A drug for preventing and treating insomnia, anxiety and depression is provided, which includes N1-dihydrocaffeoyl-N10-caffeoylspermidine.

Further, the dosage form is an oral dosage form, a topical patch or an injectable dosage form as permitted in pharmacology.

Further, a single application dose of N1-dihydrocaffeoyl-N10-caffeoylspermidine is 2-100 mg/kg.

Further, a single application dose of N1-dihydrocaffeoyl-N10-caffeoylspermidine is 2-40 mg/kg.

Further, a single application dose of N1-dihydrocaffeoyl-N10-caffeoylspermidine is 5-10 mg/kg.

A health product for improving insomnia, anxiety, and depression is provided, which includes N1-dihydrocaffeoyl-N10-caffeoylspermidine.

A functional food for improving insomnia, anxiety, and depression is provided, which includes N1-dihydrocaffeoyl-N10-caffeoylspermidine.

According to the above technical solutions, compared with the prior art, the disclosure has the following beneficial effects: At the dose range of 2-100 mg/kg that the experimental animals were given, N1-dihydrocaffeoyl-N10-caffeoylspermidine significantly reduced the number of activities, shortened the sleep latency and prolonged the sleep duration of mice with insomnia, as well as significantly regulated back the indicator levels of neurotransmitters such as 5-HT, 5-HIAA, GABA and Glu in the brain tissues of mice with insomnia. All of the above confirm that N1-dihydrocaffeoyl-N10-caffeoylspermidine have a sedative hypnotic effect and can be applied for the prevention and treatment of psychiatric disorders such as insomnia, anxiety and depression.

### BRIEF DESCRIPTION OF THE DRAWINGS

In order to explain the embodiments of the present disclosure or the technical solutions in the prior art more clearly, the following drawings that need to be used in the description of the embodiments or the prior art are briefly introduced. Obviously, the drawings in the following description are only embodiments of the present disclosure. For those of ordinary skill in the art, other drawings can be obtained based on the drawings disclosed without creative work.

FIG. 1 shows the effect of N1-dihydrocaffeoyl-N10-caffeoylspermidine on the number of autonomous activities of the mice after the last administration in Experiment 1 of the present disclosure (3 represents N1-dihydrocaffeoyl-N10-caffeoylspermidine; G, Z and D represent the high dose group, medium dose group and low dose group, respectively; compared with the model group, *p<0.01, **p<0.001).

FIG. 2 shows the effect of N1-dihydrocaffeoyl-N10-caffeoylspermidine on the sleep duration of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, ^{#}p<0.05; compared with the model group, *p<0.05, *p<0.01, **p<0.001).

FIG. 3 shows the effect of N1-dihydrocaffeoyl-N10-caffeoylspermidine on the sleep latency of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, ^{###}p<0.001; compared with the model group, *p<0.05, **p<0.001).

FIG. 4 shows the effect of N1-dihydrocaffeoyl-N10-caffeoylspermidine on 5-HT in brain tissues of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, ^{###}p<0.001; compared with the model group, *p<0.05, *p<0.01, **p<0.001).

FIG. 5 shows the effect of N1-dihydrocaffeoyl-N10-caffeoylspermidine on 5-HIAA in brain tissues of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, ^{###}p<0.001; compared with the model group, *p<0.01, **p<0.001).

FIG. 6 shows the effect of N1-dihydrocaffeoyl-N10-caffeoylspermidine on GABA in brain tissues of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, _{##}p<0.01; compared with the model group, *p<0.01, **p<0.001).

FIG. 7 shows the effect of N1-dihydrocaffeoyl-N10-caffeoylspermidine on Glu in brain tissues of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, ^{#}p<0.05; compared with the model group, *p<0.05, **p<0.001).

FIG. 8 shows the effect of N1-dihydrocaffeoyl-N10-caffeoylspermidine on Glu/GABA ratio in brain tissues of the mice in Experiment 1 of the present disclosure (3 represents N1-dihydrocaffeoyl-N10-caffeoylspermidine; G, Z and D represent the high dose group, medium dose group and low dose group, respectively; compared with the normal control group, ##p<0.01; compared with the model group, *p<0.05, **p<0.001).

### DETAILED DESCRIPTION OF THE EMBODIMENTS

Technical solutions of the present disclosure will be clearly and completely described below with reference to the accompanying drawings in the embodiments of the present disclosure. Obviously, the described embodiments are only part of the embodiments of the present disclosure, not all of them. Based on the embodiments of the disclosure, all other embodiments made by those skilled in the art without sparing any creative effort should fall within the protection scope of the disclosure.

The N1-dihydrocaffeioyl-N10-caffeioylspermidine used in the following embodiments are all compounds shown in formula (1) above and can be obtained through commercial or experimental preparation.

The required reagents for the present disclosure are conventional experimental reagents purchased from commercial channels; the experimental methods not mentioned are conventional and will not be repeated here.

### Embodiment 1

A drug for preventing and treating of insomnia, anxiety and depression is provided, in the form of an injectable dosage form, including N1-dihydropcaffeioyl-N10-caffeioylspermidine, wherein the single application dose of N1-dihydrocaffeoyl-N10-caffeoylspermidine is 2 mg/kg.

### Embodiment 2

A health product for improving insomnia, anxiety and depression is provided, in the form of an oral health product, including N1-dihydrocaffeoyl-N10-caffeoylspermidine, wherein the single application dose of N1-dihydrocaffeoyl-N10-caffeoylspermidine is 100 mg/kg.

### Embodiment 3

A drug for preventing and treating of insomnia, anxiety and depression is provided, in the form of an oral dosage form, including N1-dihydrocaffeoyl-N10-caffeoylspermidine, wherein the single application dose of N1-dihydrocaffeoyl-N10-caffeoylspermidine is 40 mg/kg.

### Embodiment 4

A drug for preventing and treating of insomnia, anxiety and depression is provided, in the form of a topical patch, including N1-dihydrocaffeoyl-N10-caffeoylspermidine, wherein the single application dose of N1-dihydrocaffeoyl-N10-caffeoylspermidine is 10 mg/kg.

### Embodiment 5

A drug for preventing and treating of insomnia, anxiety and depression is provided, in the form of an oral dosage form, including N1-dihydrocaffeoyl-N10-caffeoylspermidine, wherein the single application dose of N1-dihydrocaffeoyl-N10-caffeoylspermidine is 10 mg/kg.

### Experiment 1

The following animal experiment further illustrates the effects of Embodiment 1 to Embodiment 5 above:

I. Design of Animal Experiment

The pharmacodynamic evaluation of N1-dihydrocaffeoyl-N10-caffeoylspermidine for improving sleep effect was performed in a mouse insomnia model induced by injection of para-chlorophenylalanine (PCPA). The sedative hypnotic effect of N1-dihydrocaffeoyl-N10-caffeoylspermidine in mice with insomnia were evaluated by behavioral evaluation methods, such as recording of the number of autonomous activities, recording of body weight and inducing sleep experiments in mice with pentobarbital sodium. Brain tissues was obtained to determine the levels of neurotransmitters 5-HT, 5-HIAA, GABA and Glu, and to explore the possible mechanism of the sleep improving effect of N1-dihydrocaffeoyl-N10-caffeoylspermidine.

II. Process of Experiment

1. Experimental Animals

Healthy SPF grade male ICR mice (18-22 g) were obtained, which were provided by the animal experimental center of Ningxia Medical University (laboratory animal license number SCXK (Ningxia Medical University) 2020-0001). The mice were adaptively housed in a standard SPF grade laboratory with the same animal housing conditions for each group: ambient temperature (25° C.), relative humidity (50%-60%). The mice were free access to food and water.

2. Experimental Method

The ICR mice were adaptively fed for 3 days before modeling, and the day before modeling, they were randomly divided into 6 groups, that is, normal control group, model group, positive drug group (melatonin), N1-dihydrocaffeoyl-N10-caffeoylspermidine groups (low dose group 2 mg/kg/d, medium dose group 40 mg/kg/d, and high dose group 100 mg/kg/d), and 10 mice per group. After adaptive feeding, for 2 days of continuous modeling, the normal control group was given normal saline (0.1 mL/10 g/d), and the remaining groups received an intraperitoneal injection of PCPA suspension (400 mg/kg/d). Within 28-30 h after injection, the mice showed non-stop daytime activity, reduced feeding and increased aggression, indicating successful modeling.

After the success of modeling, the normal control group and the model group were administrated with normal saline (0.1 mL/10 g/d), and the dosage administered in the positive drug group was 10 mg/kg/d, and the dosages administered in N1-dihydrocaffeoyl-N10-caffeoylspermidine groups were 2, 40 and 100 mg/kg/d, respectively. The mice in each group were administered sequentially from 8:00 am to 12:00 am daily for 7 days.

3. Observation of Indicators

(1) Number of autonomous activities: after grouping as above, the number of autonomous activities of mice in 5 min was recorded after successful modeling and half an hour after administration on the 7th day, respectively. Mice were acclimatized into the apparatus for 3 min before each recording.

(2) Body weight: the body weight of mice in each group was recorded before modeling, after modeling, on the 3rd, 7th day of drug administration, respectively.

(3) Righting reflex experiment to determine sleep duration: one hour after administration on the 7th day, mice in each group were intraperitoneally injected with pentobarbital sodium suspension (50 mg/kg), and the sleep latency and sleep duration of mice were recorded. (Sleep latency was recorded from the end of the intraperitoneal injection until the disappearance of the mouse righting reflex for more than 1 min; time from falling asleep to the recovery of the mouse righting reflex was recorded as sleep duration.)

4. Determination of Levels of Neurotransmitters

After administration for 7 days, the mice were sacrificed by cervical dislocation, the brains were removed by decapitation on an ice table, ground evenly with a tissue homogenizer, and normal saline was added to make 10% brain tissue homogenate. The homogenate was centrifuged at 3000 r/min for 15 min at 4° C., then supernatants were collected, and levels of neurotransmitters 5-HT, 5-HIAA, Glu, and GABA were determined by ELISA.

5. Data Processing

In the data processing of the present disclosure, SPSS 18.0 was used for data analysis and statistics, the obtained results were all expressed as the mean standard deviation (X±S), the comparison of differences among groups was performed using one-way ANOVA, and the differences were statistically significant with p<0.05 as the standard.

III. Experimental Result

1. Effect on Number of Autonomous Activities of Mice

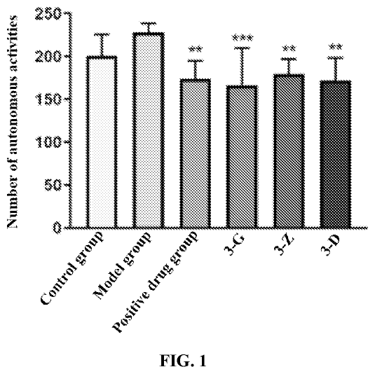

The results of the effect of N1-dihydrocaffeoyl-N10-caffeoylspermidine on the number of autonomous activities of mice are shown in Table 1 and FIG. 1.

TABLE 1
Effect of N1-dihydrocaffeoyl-N10-caffeoylspermidine
on number of autonomous activities of mice

Number of autonomous activities

| | Before | After last |
|---|---|---|
| Group | administration | administration |
| Normal control group | 190 ± 30.85 | 200 ± 25.30 |
| Model group | 255 ± 12.37^{###} | 228 ± 10.43 |
| Positive drug group | 267 ± 15.15^{###} | 174 ± 20.81*** |
| N1-dihydrocaffeoyl-N10- | 248 ± 18.38^{###} | 167 ± 43.35*** |
| caffeoylspermidine | | |
| group at high dose | | |
| N1-dihydrocaffeoyl-N10- | 246 ± 16.13^{###} | 179 ± 17.43*** |
| caffeoylspermidine | | |
| group at medium dose | | |
| N1-dihydrocaffeoyl-N10- | 249 ± 22.30^{###} | 172 ± 26.39*** |
| caffeoylspermidine | | |
| group at low dose | | |

Note:
^{#}represents a comparison between the model group and the normal control group before administration, ^{#}p < 0.05, ^{##}p < 0.01, ^{###}p < 0.001; *represents a comparison between the mice after last administration and those corresponding before administration, *p < 0.05, **p < 0.01, ***p < 0.001.

For the mice before administration, compared with the normal control group (190±30.85), the insomnia groups induced by PCPA showed a significant increase in autonomous activity frequency (p<0.01). For the mice after last administration, compared with those before administration, there was no significant change in the number of autonomous activities of the normal control group and model group mice, while the number of autonomous activities in the positive drug group and N1-dihydrocaffeoyl-N10-caffeoylspermidine groups at all doses significantly decreased (p<0.001).

2. Effect on Body Weight of Mice

The results of the effect of N1-dihydrocaffeioyl-N10-caffeioylspermidine on body weight of mice are shown in Table 2.

TABLE 2
Effect of N1-Dihydrocaffeioyl-N10-caffeioylspermidine on body weight of mice

| | Body weight (g) | | | |
|---|---|---|---|---|
| | Before | After | 3rd day of | 7th day of |
| Group | molding | molding | administration | administration |
| Normal control group | 25.56 ± 2.82 | 29.46 ± 2.07^{##} | 31.71 ± 2.27*** | 32.45 ± 1.34 |
| Model group | 27.42 ± 2.53 | 27.23 ± 2.64 | 26.50 ± 3.39 | 29.95 ± 4.07 |
| Positive drug group | 26.51 ± 2.23 | 26.26 ± 3.39 | 31.33 ± 4.21*** | 33.67 ± 3.80** |
| N1-dihydrocaffeoyl-N10- | 26.18 ± 2.78 | 26.87 ± 2.71 | | |
| caffeoylspermidine | | | 32.36 ± 2.39*** | 34.66 ± 3.00** |
| group at high dose | | | | |
| N1-dihydrocaffeoyl-N10- | 25.95 ± 1.99 | 27.33 ± 2.13 | 31.34 ± 1.76*** | 32.12 ± 1.44 |
| caffeoylspermidine | | | | |
| group at medium dose | | | | |
| N1-dihydrocaffeoyl-N10- | 26.74 ± 2.63 | 27.08 ± 3.74 | 32.02 ± 2.63*** | 31.83 ± 2.77 |
| caffeoylspermidine | | | | |
| group at low dose | | | | |

Note:
compared with before modeling, ^{#}p < 0.05, ^{##}p < 0.01, ^{###}p < 0.001;
Note:
compared with the model group, *p < 0.05, **p < 0.01, ***p < 0.01.

For the comparison between the mice before modeling and those after modeling, the body weight of mice in the normal control group gradually increased as the experiment proceeded (p<0.01), while the body weight of mice in the model group basically remained unchanged without statistical difference. On the 3rd day of administration, compared with the normal control group (31.71±2.27 g), the body weight of mice in the model group (26.50±3.39 g) was significantly reduced (p<0.001); compared with the model group, the body weight of mice in the positive drug group and N1-dihydrocaffeoyl-N10-caffeoylspermidine groups at all doses increased significantly (p<0.001). On the 7th day of administration, compared with the normal control group (32.45±1.34 g), the body weight of mice in the model group (29.95±4.07 g) was slightly reduced, but it was not statistically significant; compared with the model group, the body weight of mice in both the positive drug group and N1-dihydrocaffeoyl-N10-caffeoylspermidine group at high dose increased significantly (p<0.01).

3. Effects on Sleep Latency and Sleep Duration of Mice

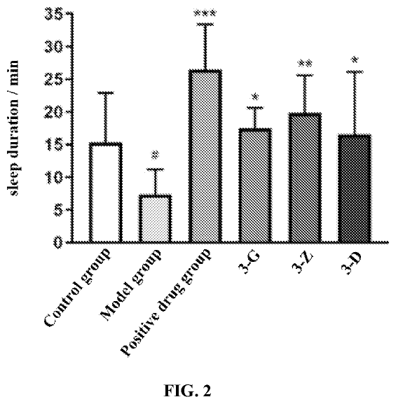

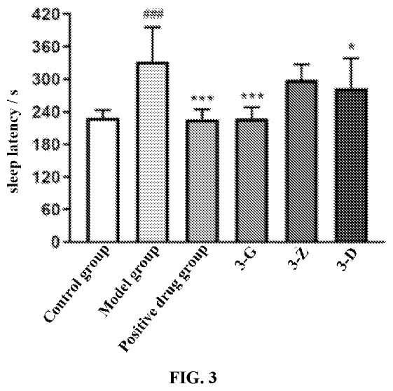

The results of the effects of N1-dihydrocaffeoyl-N10-caffeoylspermidine on sleep duration and sleep latency of mice are shown in Table 3, FIG. 2 and FIG. 3.

TABLE 3
Effects of N1-dihydrocaffeoyl-N10-caffeoylspermidine
on sleep latency and sleep duration of mice

| Group | Sleep latency (s) | Sleep duration (s) |
|---|---|---|
| Normal control group | 229.00 ± 13.70 | 15.29 ± 7.60 |
| Model group | 332.67 ± 62.76^{###} | 7.38 ± 3.81^{#} |
| Positive drug group | 226.00 ± 18.42*** | 26.45 ± 6.92*** |
| N1-dihydrocaffeoyl-N10- | 227.33 ± 21.13*** | 17.46 ± 3.15* |
| caffeoylspermidine | | |
| group at high dose | | |
| N1-dihydrocaffeoyl-N10- | 298.50 ± 29.08 | 19.87 ± 5.72** |
| caffeoylspermidine | | |
| group at medium dose | | |
| N1-dihydrocaffeoyl-N10- | 283.17 ± 55.51* | 16.56 ± 9.53* |
| caffeoylspermidine | | |
| group at low dose | | |

Note:
compared with the normal control group, ^{#}p < 0.01, ^{##}p < 0.01, ^{###}p < 0.001; compared with the model group, *p < 0.05, **p < 0.01, ***p < 0.001.

The results showed that in terms of sleep latency indicator level, compared with the normal control group (229.00±13.70 s), the sleep latency of mice in the model group (332.67±62.76 s) was significantly increased (p<0.001), indicating the success of the PCPA-induced insomnia model of mice, which caused the phenomenon of difficulty in falling asleep. Compared with the model group, the sleep latency of mice in the positive drug group and N1-dihydrocaffeoyl-N10-caffeoylspermidine groups at high and low doses was significantly reduced (p<0.05), indicating that both the positive drug melatonin and N1-dihydrocaffeoyl-N10-caffeoylspermidine can regulate the sleep latency of mice and have an improving effect on PCPA-induced insomnia of mice. In terms of sleep duration indicator levels, compared with the normal control group (15.29±7.60 min), the sleep duration of mice in the model group (7.38±3.81 min) was significantly shorter (p<0.05), indicating that the PCPA-induced insomnia model of mice was successful in shortening the sleep duration of mice. Compared with the model group, the sleep duration of mice in the positive drug group and N1-dihydrocaffeoyl-N10-caffeoylspermidine groups at all doses increased significantly (p<0.05), indicating that the positive drug melatonin and N1-dihydrocaffeoyl-N10-caffeoylspermidine can increase the sleep duration of mice and have an improving effect on PCPA-induced insomnia of mice.

In conclusion, mice with insomnia showed non-stop daytime activity, reduced feeding and sleep, resulting in the increased number of autonomous activities (p<0.001), longer sleep latency (p<0.001), shorter sleep duration (p<0.05), and decreased body weight gain rate of mice. The N1-dihydrocaffeoyl-N10-caffeinylspermidine groups at different doses reduced the number of autonomous activities, shortened sleep latency, increased sleep duration, and improved the decrease of body weight gain rate in mice with insomnia caused by PCPA to different degrees. The above data indicate that N1-dihydrocaffeoyl-N10-caffeoylspermidine has a significant sedative hypnotic effect.

4. Effect on Levels of Monoamine Neurotransmitters of Mice

The results of the effect of N1-dihydrocaffeoyl-N10-caffeoylspermidine on the levels of monoamine neurotransmitters are shown in Table 4.

TABLE 4
Effect of N1-dihydrocaffeoyl-N10-caffeoylspermidine
on monoamine neurotransmitters of mice

| Group | 5-HT (ng/g) | 5-HIAA (ng/g) |
|---|---|---|
| Normal control group | 322.96 ± 28.00 | 34.37 ± 3.71 |
| Model group | 238.66 ± 34.66^{###} | 29.66 ± 1.74^{###} |
| Positive drug group | 318.30 ± 34.56*** | 37.15 ± 1.56^{###} |
| N1-dihydrocaffeoyl-N10- | 302.55 ± 17.00*** | 36.61 ± 1.84*** |
| caffeoylspermidine | | |
| group at high dose | | |
| N1-dihydrocaffeoyl-N10- | 283.18 ± 9.86** | 36.69 ± 2.00*** |
| caffeoylspermidine | | |
| group at medium dose | | |
| N1-dihydrocaffeoyl-N10- | 271.37 ± 27.58* | 33.79 ± 2.51** |
| caffeoylspermidine | | |
| group at low dose | | |

Note:
compared with the normal control group, ^{#}p < 0.05, ^{##}p < 0.01, ^{###}p < 0.001; compared with the model group, **p < 0.05, ***p < 0.001.

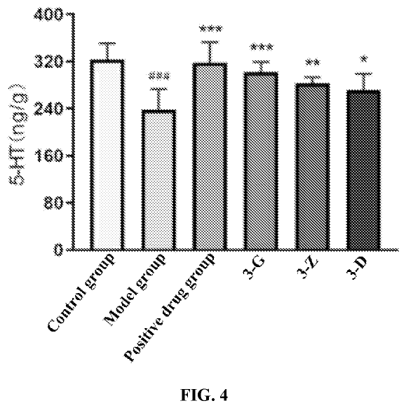

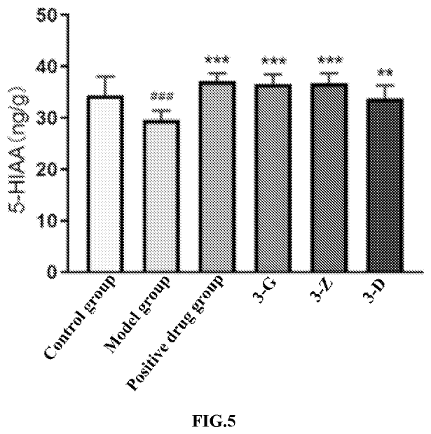

The results of the effect of N1-dihydrocaffeoyl-N10-caffeoylspermidine on 5-HT and 5-HIAA in brain tissues of mice are shown in Table 4, FIG. 4 and FIG. 5. Compared with the normal control group (322.96±28.00 ng/g), the 5-HT (238.66±34.66 ng/g) content in the brain tissues of mice in the model group was significantly reduced (p<0.001), indicating the success of the PCPA-induced insomnia model of mice. Compared with the model group, the 5-HT content in the brain tissues of mice in the positive drug group and N1-dihydrocaffeoyl-N10-caffeoylspermidine groups at all doses was significantly increased (p<0.05), and there was a certain positive dose correlation, that is, the higher the administered dose, the higher the 5-HT content. Compared with the normal control group (34.37±3.71 ng/g), the 5-HIAA (29.66±1.74 ng/g) content in the brain tissues of mice in the model group was significantly lower (p<0.001), indicating the success of the PCPA-induced insomnia model of mice. Compared with the model group, the 5-HIAA content in the brain tissues of mice in the positive drug group and the N1-dihydrocaffeoyl-N10-caffeoylspermidine groups at all doses was significantly increased (p<0.01).

In conclusion, the 5-HT and 5-HIAA contents in brain tissues of mice with insomnia were reduced (p<0.001). The N1-dihydrocaffeoyl-N10-caffeoylspermidine groups at different doses were able to increase the 5-HT and 5-HIAA contents to different degrees, and it was speculated that N1-dihydrocaffeoyl-N10-caffeoylspermidine may improve sleep by directly increasing the 5-HT and 5-HIAA contents in brain tissues of mice with insomnia.

5. Effect on Levels of Amino Acid Neurotransmitters of Mice

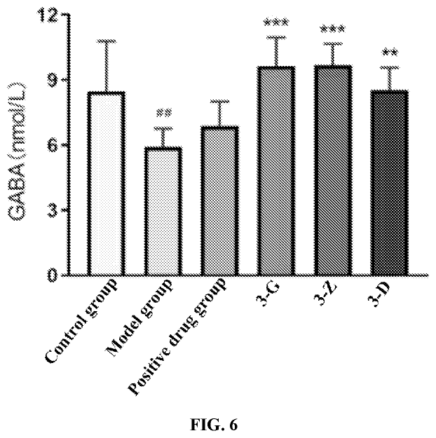

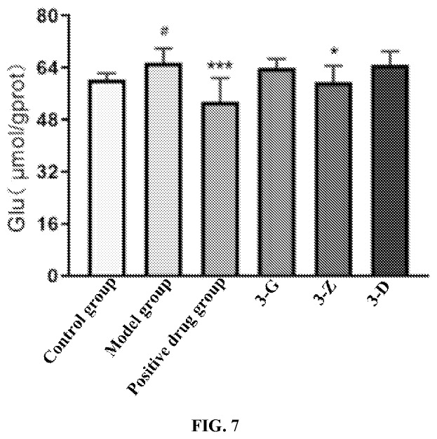

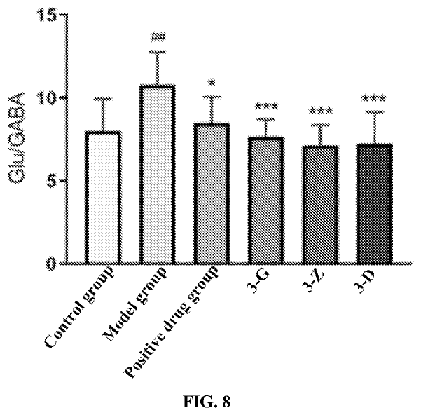

The results of the effect of N1-dihydrocaffeoyl-N10-caffeoylspermidine on the levels of amino acid neurotransmitters are shown in Table 5, FIG. 6, FIG. 7 and FIG. 8.

TABLE 5
Effect of N1-dihydrocaffeoyl-N10-caffeoylspermidine
on amino acid neurotransmitters of mice

| Group | GABA (nmol/L) | Glu (μmol/gprot) | Glu/GABA |
|---|---|---|---|
| Normal control group | 8.47 ± 2.31 | 60.31 ± 1.93 | 8.00 ± 1.94 |
| Model group | 5.94 ± 0.83^{##} | 65.56 ± 4.33^{#} | 10.79 ± 1.96^{##} |
| Positive drug group | 6.89 ± 1.11 | 53.52 ± 7.23*** | 8.51 ± 1.55* |
| N1-dihydrocaffeoyl-N10- | 9.66 ± 1.30*** | 63.90 ± 2.88 | 7.66 ± 1.03*** |
| caffeoylspermidine | | | |
| group at high dose | | | |
| N1-dihydrocaffeoyl-N10- | 9.69 ± 0.98*** | 59.58 ± 5.02* | 7.17 ± 1.20*** |
| caffeoylspermidine | | | |
| group at medium dose | | | |
| N1-dihydrocaffeoyl-N10- | 8.53 ± 1.03** | 64.84 ± 4.18 | 7.23 ± 1.90*** |
| caffeoylspermidine | | | |
| group at low dose | | | |

Note:
compared with the normal control group, ^{#}p < 0.05, ^{##}p < 0.01, ^{###}p < 0.001; compared with the model group, **p < 0.01, ***p < 0.001.

The results of the effect of N1-dihydrocaffeoyl-N10-caffeoylspermidine on GABA, Glu and Glu/GABA values in brain tissues of mice are shown in Table 5, FIG. 6, FIG. 7 and FIG. 8. Compared with the normal control group (8.47±2.31 nmol/L), the GABA (5.94±0.83 nmol/L) content in the brain tissues of mice in the model group was significantly lower (p<0.01), indicating that the GABA content in the brain tissues of PCPA-induced mice with insomnia was reduced. Compared with the model group, the GABA (6.89±1.11 nmol/L) content in the brain tissues of mice in the positive drug group was increased, but there was no statistically significant. The GABA content in the brain tissues of mice in N1-dihydrocaffeoyl-N10-caffeoylspermidine groups at different doses was significantly increased (p<0.01). Compared with the normal control group (60.31±1.93 μmol/gprot), Glu (65.56±4.33 μmol/gprot) content in the brain tissues of mice in the model group was significantly increased (p<0.05), indicating an increase in Glu content in the brain tissues of PCPA-induced mice with insomnia. Compared with the model group, the Glu (53.52±7.23 μmol/gprot) content in the brain tissues of mice in the positive drug group was significantly decreased (p<0.001). The Glu content in the brain tissues of mice N1-dihydrocaffeoyl-N10-caffeoylspermidine group at medium dose was significantly decreased (p<0.01). Compared to the normal control group (8.00±1.94), the Glu/GABA value (10.79±1.96) in the brain tissues of mice in the model group were significantly increased (p<0.01). Compared with the model group, the Glu/GABA values in the brain tissues of mice in the positive drug group and N1-dihydrocaffeoyl-N10-caffeoylspermidine groups at different doses were significantly reduced (p<0.05).

In conclusion, the GABA content in brain tissues was decreased (p<0.01), and Glu content and Glu/GABA values were increased (p<0.05) in mice with insomnia. The N1-dihydrocaffeoyl-N10-caffeoylspermidine groups at all doses were able to increase GABA content and decrease Glu/GABA values in brain tissues of mice with insomnia. It is speculated that N1-dihydrocaffeoyl-N10-caffeoylspermidine may improve sleep by increasing GABA content and decreasing Glu/GABA values.

During the whole animal administration process, no mice died and no abnormalities occurred in the appearance and behavior of the mice.

Each embodiment in this specification is described in a progressive manner, with each embodiment focusing on what is different from the other embodiments, and the same similar parts between each embodiment can be referred to each other.

The above description of the disclosed embodiments enables a person skilled in the art to implement or use the disclosure. A variety of modifications to these embodiments will be apparent to those skilled in the art, and the general principles defined herein can be implemented in other embodiments without departing from the spirit or scope of the present disclosure. Thus, the disclosure will not be limited to these embodiments shown herein, but will be subject to the widest scope consistent with the principles and novel features disclosed herein.

What is claimed is:

1. An application of N1-dihydrocaffeioyl-N10-caffeioylspermidine in a preparation of a drug for preventing and treating insomnia, anxiety and depression, or in a preparation of a health product or a functional food for improving insomnia, anxiety and depression.

2. The application of claim 1, wherein a number of autonomous activities of mice with insomnia can be reduced.

3. The application of claim 1, wherein sleep latency can be shortened.

4. The application of claim 1, wherein sleep duration can be prolonged.

5. The application of claim 1, wherein levels of transmitters 5-HT, 5-HIAA, GABA and Glu in brain tissues can be regulated back.

6. A drug for preventing and treating insomnia, anxiety and depression, characterized by comprising N1-dihydrocaffeoyl-N10-caffeoylspermidine.

7. The drug of claim 6, wherein a dosage form is an oral dosage form, a topical patch or an injectable dosage form as permitted in pharmacology.

8. A health product for improving insomnia, anxiety, and depression, characterized by comprising N1-dihydrocaffeoyl-N10-caffeoylspermidine.

9. A functional food for improving insomnia, anxiety, and depression characterized by comprising N1-dihydrocaffeoyl-N10-caffeoylspermidine.
